(19) United States
(12) Patent Application Publication
(10) Pub. No.: US 2021/0308407 A1
(43) Pub. Date: Oct. 7, 2021

(54) MEDICAL ULTRAVIOLET-C REMOTE RESPIRATOR

(71) Applicant: Armor Respiration, Inc., Hingham, MA (US)

(72) Inventor: Lisa Fitzgerald, Leesburg, FL (US)

(21) Appl. No.: 16/839,187

(22) Filed: Apr. 3, 2020

(51) Int. Cl. A61M 16/10; A61M 16/06; A61M 16/20; A61L 2/00; A61L 2/26

(52) U.S. Cl. CPC A61M 16/1065; A61M 16/0605; A61M 16/0666; A61M 16/208; A61L 2/0047; A61L 2/26; A61L 2202/22; A61M 2202/203; A61M 2202/0208; A61M 2205/8237; A61M 2209/088; A61L 2202/16; A61M 2202/206

(57) ABSTRACT

An ultraviolet-C respirator mask that filters and destroys viruses by means of a HEPA type filter and a plurality of UVC LEDs projected on the filter. The filter element is worn on the garment of the user by means of an integral apparel clip and connected to the face mask through a breathing hose. This configuration allows easy replacement of the HEPA filter element and/or the facemask for considerations of hygiene. Filter element life is increased due to location further from contaminated field. Hygiene for the user is improved due to the collecting media being further from the user's face.

### TECHNICAL FIELD

The present invention relates generally to the field of respirators, and more particularly, to a medical respirator with an integral ultraviolet-C light sanitizer worn or clipped to a garment with an extension hose connected to a face mask.

### BACKGROUND

A respirator is a device to protect the user from inhaling hazardous atmosphere, including particulate matter such as dust and airborne microorganisms (e.g., bacteria and/or virus particles) commonly used in the healthcare industry. Respirators are typically worn in a clinical setting by a clinician to reduce the possibility of nosocomial infections between clinician and patient (i.e., transmission of virus(es) and/or bacteria pass between the physician and patient).

N95 masks/respirators are the most commonly used respirators within the clinical setting. N95 respirators are disposable face masks with an N95-specified face piece (e.g., filters that block particulate matter of 0.3 μm or greater), N95 respirators are secured to the wearer's head with straps, a doth harness, or some other method. These respirators are discarded when they become unsuitable for further use due to considerations of hygiene, excessive resistance, or physical damage. Due to the recent COVID-19 pandemic, N95 respirators are in short supply and great demand, with many clinicians repeatedly reusing the same N95 for multiple works shifts. Many clinicians have further resulted in attempting to sanitize these disposable respirators (e.g., with UV or cleaning chemicals), which may in itself be unsafe due to the fact that these respirators are not particularly durable and were manufactured for disposable, single use.

In addition to the N95 respirators, other types of respirator masks used for bacteria and virus control are full-face types that cover the entire face to protect the eyes and facial area. These tasks also employ a filtration system that impedes viruses and bacteria through a mechanism called diffusion. The viruses and bacteria that pass through the filter media, especially those below 100 nm in diameter, collide with gas molecules, and are impeded and delayed. An electrostatic charge then holds them onto the filter surface.

It should be noted that each type of respirator discussed immediately above is currently in short supply and high-demand to the COVID-19 outbreak/pandemic. Furthermore, production of the above respirators is not a trivial task. Thus, an alternative respirator that is highly effective versus microorganism transmission (e.g., COVID-19) and that further greatly prevents nosocomial infections in a clinical setting that is also easily produced/manufactured is greatly needed.

### SUMMARY

In view of the above mentioned need, thus disclosed is a medical ultraviolet-C remote respirator, which is an improved implementation of the existing respirator mask such as the N95 mask and is configured for reuse thus avoiding problems currently seen with attempting to sanitize disposable N95 respirators. The filter element is worn remote, in a housing on or in the user's garment. The housing design allows use of a replaceable HEM (high-efficiency particulate absorbing) filter, which can also filter out approximately 98% or greater or 99% or greater particulate matter having a diameter of 0.3 μm or greater. It should be noted that the largest airborne particulate matter is approximately 10 μm, and thus, the filtration range ranges approximately from 0.3 μm to 10 μm. The advantages of this configuration is that it relocates the toxic collecting filter away from the user's face and allows use of a filter with a larger surface area which provides easier breathing. This device can be used longer due to collected viruses and bacteria not being in proximity to the clinician's or patient's face or upper body. This device can also advantageously be easily cleaned and fitted with a new HEPA filter as required. Additional protection of the HEPA filter is achieved if the device is worn under clothing, thereby increasing the life of the filter. Separately, the plastic face mask and hose can be easily cleaned and/or replaced between patients, as required, for considerations of hygiene.

Having the filter element in a remote housing allows the addition of a plurality of ultraviolet-C (UVC) 280 nm LEDs (light-emitting diodes) in proximity to the HEPA filter. In this configuration, the UVC LEDs are projected to the back side (user side) of the HEPA filter to reduce or eliminate contamination of viruses that may migrate through the filter. In certain aspects, the UVC LEDs may be configured to emit light at any wavelength within the UVC spectrum (i.e., from approximately 100 to approximately 280 nm) to further sanitize desired surfaces.

Each of the plurality of UVC LEDs is powered by a lithium-ion battery integral to the device, and is rechargeable by means of a charging port also integral to the device. An ON-OFF switch activates the UVC LEDs. Indicator LEDs show the status of the battery charge and the UVC LED activation. The low battery detector will flash the low battery indicator and sound an audible alarm. In other embodiments, a pressure detector will monitor the device to check that all valves and hoses are operational.

UVC is ultraviolet radiation within a specifically defined wavelength (100 to 280 nm) that has virucidal, bactericidal and germicidal properties and can destroy the ability of micro-organisms to replicate by invoking irreparable DNA damage therein. Wavelengths in the UVC range are especially damaging to, for example bacteria and viruses, because these wavelengths may invoke DNA breaks (e.g., single stranded or double stranded DNA break) and/or may further created thymine dimers and/or other nucleic acid damage that may create DNA mispairing and further invoke DNA mismatch repair and DNA base excision repair. However, when bacteria and/or viruses are exposed to UVC for prolonged periods, irreparable damage occurs and ultimately leads to bacterial death and/or viral/pathogenic inactivity.

HEPA was commercialized in the 1950s, and the original term became a registered trademark and later a generic term for highly efficient filters. Common standards require that a HEPA air filter must remove least 99.95% (European Standard) or 99.97% (ASME, U.S. DOE) of particles whose diameter is equal to 0.3 μm. It should be noted that the largest airborne particulate matter is approximately 10 μm, and thus, the filtration range ranges approximately from 0.3 μm to 10 μm. This standard meets or exceeds the standard for the typical N95 mask in common use today. In certain aspects, the HEPA filter used within the disclosed device may be interchangeable with, for example, the commercially available HEPA filters currently used in various vacuums intended for consumer use (i.e., home use/non-industrial settings).

A breathing hose is connected between the housing with the HEPA filter and the face mask. The face mask is connected to this hose by means of an air-tight connector common to many types of commercially-available masks, including masks that cover the nose and mouth and masks that cover the entire face.

This device has 3 modes of operation, as follows: (1) Inhale outside air and exhaust through the UVC and HEPA (2) Inhale and exhale through the UVC and HEPA, and (3) Inhale through the UVC and HEPA and exhale to the outside air. Air flow through the device is controlled by a plurality of valves, two exhaust valves in the face mask and one in the hose. The hose valve is in the inlet port of the HEPA filter assembly to block exhaust air back to the filter. In case (1), exhaust valves in the face mask are installed to be configured as inputs, and the hose valve is installed to input to the filter. In case (2), all the valves are removed and blocking plugs (not shown) are installed in the exhaust ports on the mask. In case (3), exhaust valves in the face mask are installed to be outputs and the hose valve is installed to output from the filter. All valves are user-configurable.

This device is mechanically-configured and sealed to allow chemical sterilization by dip or spray.

In other embodiments of this invention, the UVC light assembly may not be included and the device will function as a HEPA filter mask only.

In certain aspects disclosed is a reusable respirator comprising: (a) a mask having an inlet (32) for airtight connection to a flexible hose and an exhaust valve positioned thereon, the mask is configured to securely attach and seal to a user's face and to emit (e.g., only emit) from the exhaust valve (4) carbon dioxide or other gases exhaled from the user during respiration and to receive (e.g., only receive) oxygen through the inlet of the mask when the user inhales during respiration; (b) a flexible hose having two spaced apart ends with the first end of the flexible hose having an airtight connection with the inlet of the mask and a second end of the flexible hose having an airtight connection with an outlet of a filter assembly; and (c) a filter assembly positioned remotely from the mask, the filter assembly comprising: (i) an enclosed housing with an inlet in contact with an external atmosphere and/or environment having oxygen therein and an outlet (5) in airtight connection with the flexible hose, the inlet and outlet of the enclosed housing being in fluid connection with one another; (ii) a replaceable filter that is securely positioned over (e.g., completely over) the inlet of the enclosed housing such that oxygen from an external environment and/or atmosphere passes through the replaceable filter internally into the enclosed housing towards the outlet of the enclosed housing during respiration of the user, the replaceable filter configured to filter and/or remove and/or least 99.95% of particles having greater than or equal to 0.3 μm diameter when passing oxygen internally into the enclosed housing from the external environment and/or atmosphere; and (iii) an ultraviolet C (UVC) light source internally secured within the enclosed housing and positioned directly above the replaceable filter such that the UVC light source, while in operation, sanitizes the filter and/or oxygen passing through the filter and flowing towards the outlet of the enclosed housing by emitting light within the UVC wavelength. It should be noted that the largest airborne particulate matter is approximately 10 and thus, the filtration range of the reusable filter ranges approximately from 0.3 μm to 10 μm.

In certain aspects, a connection and/or contact interface between the replaceable filter and inlet of the enclosed housing are substantially sealed to prevent airleaks in and/or around the replaceable filter to prevent unfiltered oxygen from entering internally into the enclosed housing from the external environment and/or atmosphere.

In certain aspects, the enclosed housing is openable and closable.

In certain aspects, the enclosed housing further comprises a securing member positioned thereon that is configured to secure the enclosed housing to the respirator user.

In certain aspects, the UVC light source is operably connected to a power source. In certain aspects, the UVC light source includes at least one UVC LED within the enclosed housing. In certain aspects, the UVC light source includes a plurality of UVC LEDs that are axially aligned with one another within the enclosed housing. In each of these aspects, the UVC LEDs are positioned directly above the reusable filter as well as being positioned directly adjacent to and/or surrounding the internal airflow/oxygen flow pathway between the inlet/filter of the enclosed housing that is in fluid communication with the outlet of the enclosed housing.

In certain aspects, the power source is rechargeable.

In certain aspects, the power source is positioned internally within the enclosed housing.

In certain aspects, the replaceable filters are interchangeable with a vacuum high-efficiency particulate absorbing (HEPA) filter.

In certain aspects, a reusable respirator assembly comprising: (a) a mask having an inlet configured for an airtight connection to a flexible hose and an exhaust valve positioned thereon, (b) a flexible hose having two spaced apart ends with the first end of the flexible hose configured for an airtight connection with the inlet of the mask and a second end of the flexible hose configured for an airtight connection with an outlet of a filter assembly; and (c) a filter assembly configured for remote positioning relative to the mask, the filter assembly comprising: (i) an enclosed housing with an inlet configured for contact with an external atmosphere and/or environment having oxygen therein and an outlet configured for an airtight connection with the flexible hose, the inlet and outlet of the enclosed housing being in fluid connection with one another; (ii) a replaceable filter configured for secure positioning and/or attachment completely over the inlet of the enclosed housing such that, when assembled, oxygen from an external environment and/or atmosphere passes through the replaceable filter internally into the enclosed housing towards the outlet of the enclosed housing during respiration of the user, the replaceable filter configured to filter and/or remove and/or least 99.95% of particles having greater than or equal to 0.3 μm diameter when passing oxygen internally into the enclosed housing from the external environment and/or atmosphere; and (iii) an ultraviolet C (UVC) light source internally secured within the enclosed housing and configured for positioning directly above the replaceable filter, when fully assembled, such that the UVC light source to sanitize the filter and/or oxygen passing through the filter and flowing towards the outlet of the enclosed housing by emitting light within the UVC wavelength. It should be noted that the largest airborne particulate matter is approximately 10 μm and thus, the filtration range of the reusable filter ranges approximately from 0.3 μm to 10 μm

In certain aspects, the enclosed housing of the respirator assembly is configured for a substantially sealed connection and/or contact interface between the replaceable filter and inlet of the enclosed housing, when assembled, to prevent airleaks in and/or around the replaceable filter to prevent unfiltered oxygen from entering internally into the enclosed housing from the external environment and/or atmosphere.

In certain aspects, the enclosed housing of the respirator assembly is openable and closable.

In certain aspects, the enclosed housing of the respirator assembly further comprises a securing member positioned thereon that is configured to secure the enclosed housing to the respirator user.

In certain aspects, the UVC light source of the respirator assembly is operably connected to a power source.

In certain aspects, power source of the respirator assembly is configured for recharging.

In certain aspects, the power source of the respirator assembly is positioned internally within the enclosed housing.

In certain aspects, the replaceable filters of the respirator assembly are interchangeable with a vacuum high-efficiency particulate absorbing (HEPA) filter.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention and are intended to provide an overview or framework for understanding the nature and character of the invention as it is claimed. The accompanying drawings are included to provide a further understanding of the invention and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is an isometric view of the medical ultraviolet-C remote respirator;

FIG. 2 is a Left Isometric view of the medical ultraviolet-C remote respirator;

FIG. 3 is a Right Isometric view of the medical ultraviolet-C remote respirator;

FIG. 4 is a Bottom Isometric view of the medical ultraviolet-C remote respirator;

FIG. 5 is a Left view of the medical ultraviolet-C remote respirator;

FIG. 6 is a Front view of the medical ultraviolet-C remote respirator;

FIG. 7 is a Section view FIG. 6;

FIG. 8 is another Section view FIG. 6;

FIG. 9 is a Top view of the medical ultraviolet-C remote respirator;

FIG. 10 is a Bottom view of the medical ultraviolet-C remote respirator;

FIG. 11 is a Right view of the medical ultraviolet-C remote respirator;

FIG. 12 is a Back view of the medical ultraviolet-C remote respirator;

FIG. 13 is an Exploded view of the medical ultraviolet-C remote respirator;

FIG. 14 is a Top Isometric view of the UV printed circuit board assembly;

FIG. 15 is a Bottom Isometric view of the UV printed circuit board assembly;

FIG. 16 is an Isometric view of the mask assembly;

FIG. 17 is an Exploded view of the mask valve assembly;

FIG. 18 is an Isometric view of the hose assembly;

FIG. 19 is a Exploded view of the hose valve assembly, and

FIG. 20 is another Isometric view of the medical ultraviolet-C remote respirator.

### DETAILED DESCRIPTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be thorough and complete, and will fully convey the scope of the invention and enable one of ordinary skill in the art to make, use and practice the invention. Like reference numbers refer to like elements throughout the various drawings.

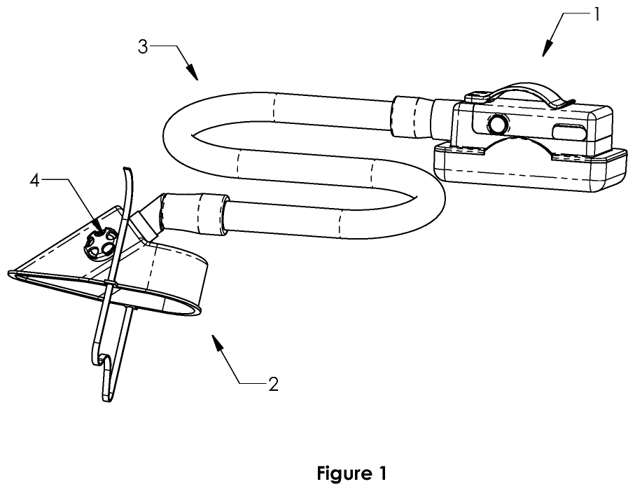

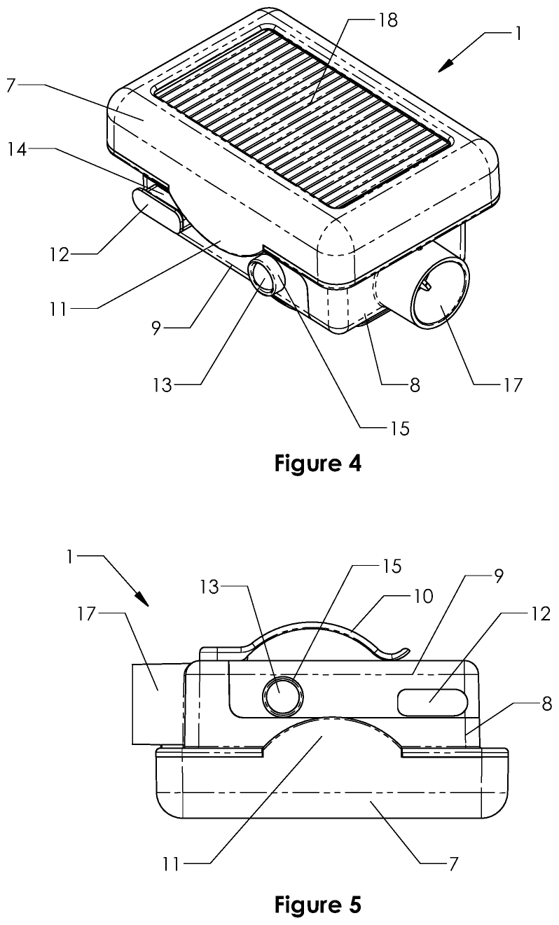

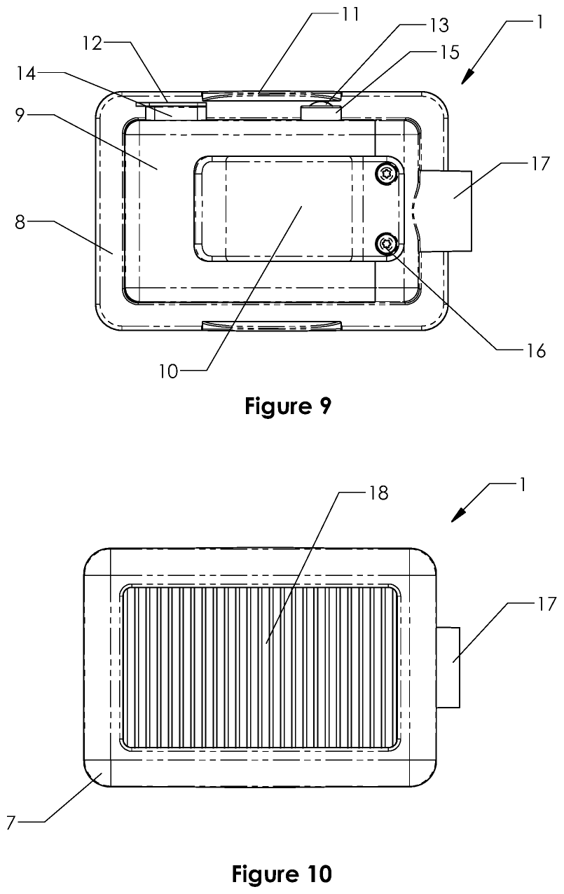

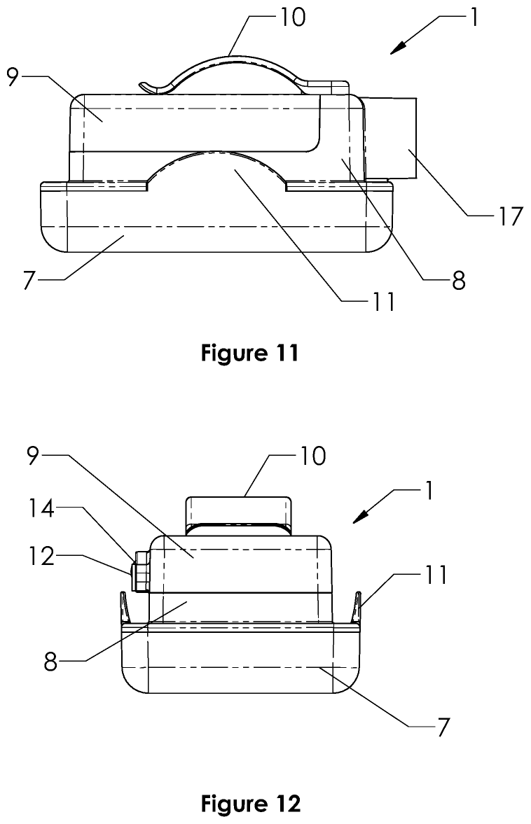

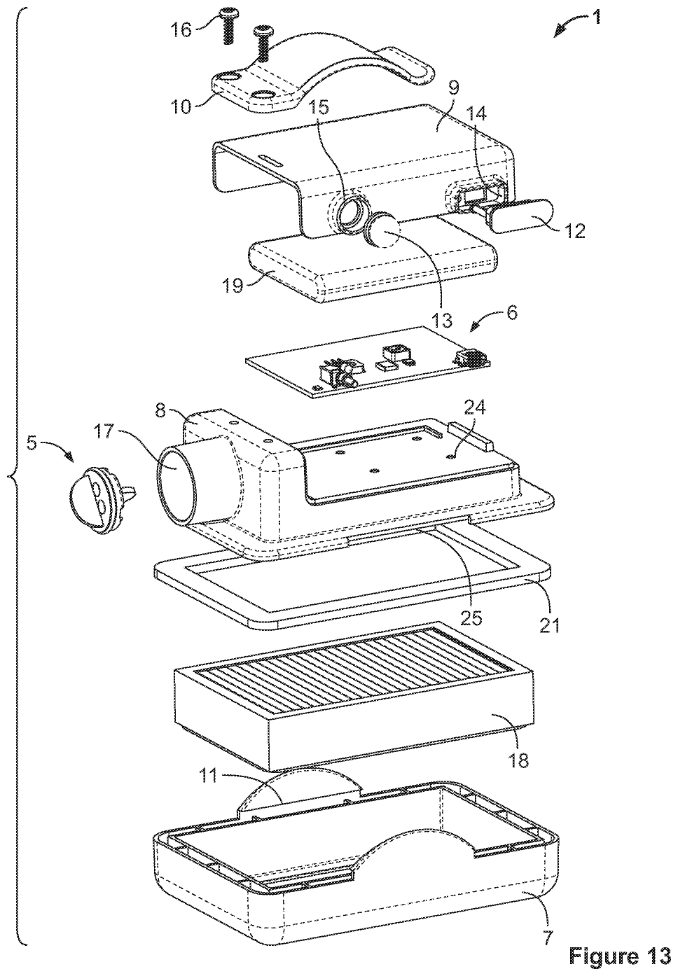

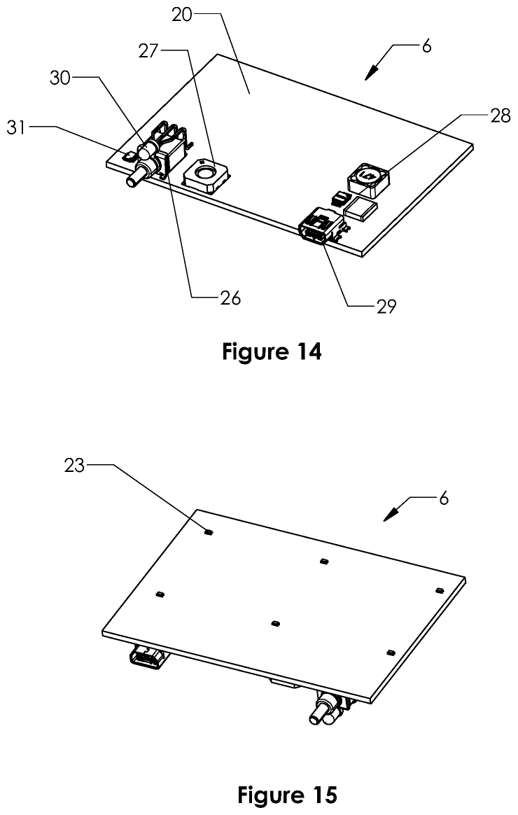

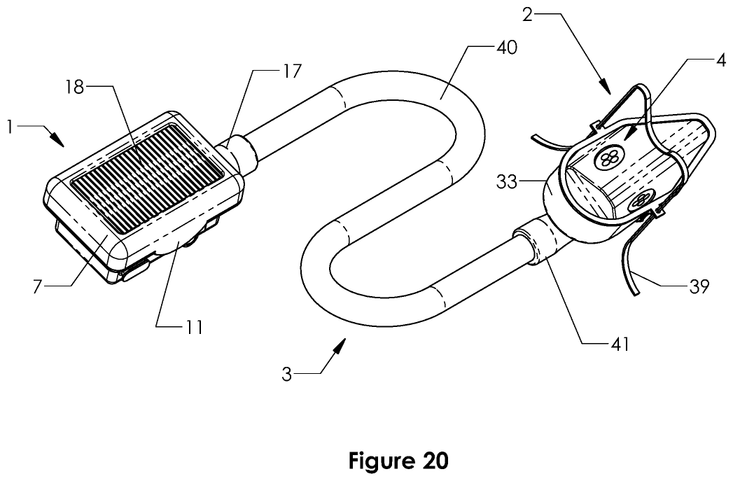

Referring to FIGS. 1, 2, 3, 4, 5, 6, 9, 10, 11, 12, 13, 14, 15, and 20, the medical ultraviolet-C remote respirator is comprised of a filter assembly 1, mask assembly 2, hose assembly 3, mask valve assembly 4, hose valve assembly 5, and a UV printed circuit board assembly 6.

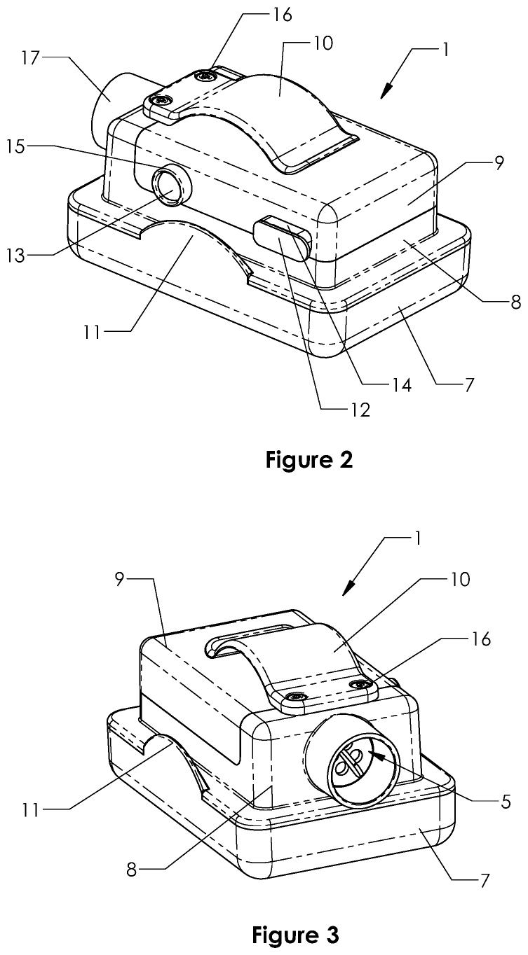

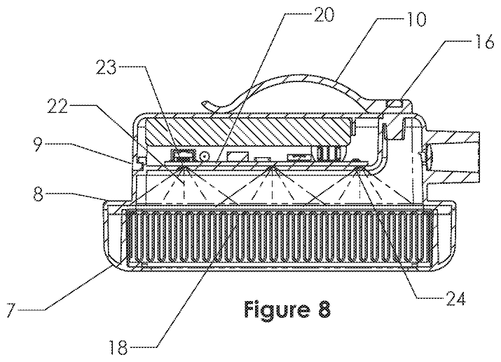

Referring to FIGS. 2, 3, 4, 5, 6, 7, 8, 9, 10, 11, 12 and 13, mainframe 8 in filter assembly 1 forms the mounting structure for the UV printed circuit board assembly 6 and the HEPA filter housing 7. The HEPA filter housing 7 holds and constrains the HEPA filter 18 by means of the filter housing clip 11, on each side of HEPA filter housing 7, engaging with the clip notch 25 on mainframe 8. As filter housing clip 11 is engaged, the HEPA filter 18 is biased against gasket 21 to form a seal between the HEPA filter 18 and flow channel 47 in mainframe 8. Gasket 21 is constructed from closed cell urethane foam with a pressure-sensitive adhesive backing to secure it to mainframe 8. Inhaled and/or exhaled air flows through HEPA filter 18 and to hose interface 17 in mainframe 8 via the flow channel 47.

Referring to FIGS. 1, 2, 3, 4, 5, 6, 7 and 15, UV printed circuit board assembly 6 is secured to mainframe 8 by silicone adhesive to seal flow channel 47, to protect from ingress of gas and liquids through the plurality of UV apertures 24 in mainframe 8. The UV printed circuit board assembly 6 forms a substrate for a plurality of UVC 280 nm LEDs 23. The 280 nm UV radiation 22 from each UVC LED is projected through the UV apertures 24 in mainframe 8, through the flow channel 47, and onto the HEPA filter 18. Any viruses in this area are exposed to this radiation.

Referring to FIGS. 2, 3, 4, 5, 6, 7, 8, 9, 11, 12, 13, 14 and 15, the plurality of UVC 280 nm LEDs 23 is powered by battery 19 secured to mainframe cover 9 by means of a pressure-sensitive adhesive. Mainframe cover 9 is captured to and held in place on mainframe 8 by apparel clip 10. Apparel clip 10 is secured to mainframe 8 by two screws 16. Apparel clip 10 is used to attach filter assembly 1 to the user's garment in normal use.

Mainframe cover 9 is sealed to mainframe 8 by a silicone adhesive bead along the contact surfaces between mainframe cover 9 and mainframe 8 to stop the ingress of gas and liquids. The power switch 26 is sealed from outside ingress by switch cover 13. Switch cover 13 is constructed from 30 durometer translucent urethane rubber to allow the switch 26 to be depressed and not break the waterproof seal. Also, the status LED 30 is visible through the translucent switch cover 13. Switch protection ring 15, an integral feature of mainframe cover 9, forms a fence around switch cover 13 so power switch 26 cannot be actuated by accident. Battery-charging connector 29 aperture is also sealed from outside ingress by means of an interfering engagement between charge port cover 12 and seal boss 14, an integral feature of mainframe cover 9.

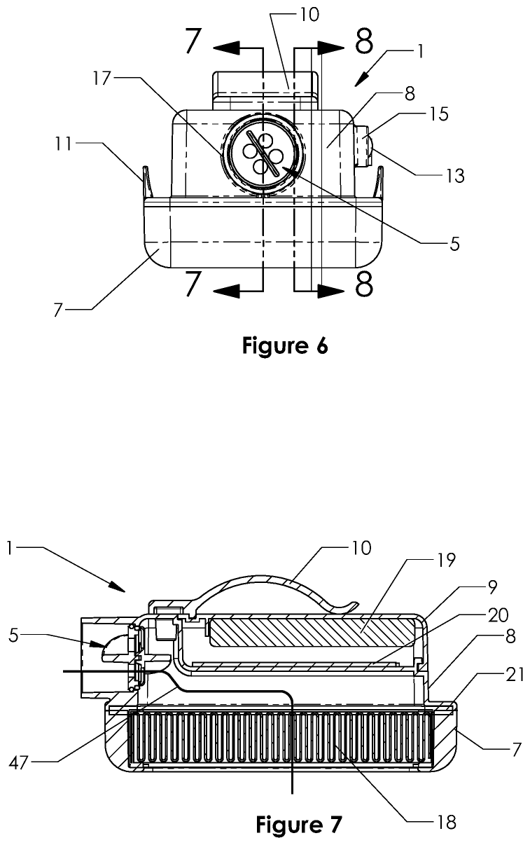

Referring to FIGS. 7, 8, 13, 14, and 15, UV printed circuit board assembly 6 is comprised of a printed circuit board 20, a plurality of UVC 280 nm LEDs 23, battery charging connector 29, boost circuit 28, audio transducer 27, power switch 26, status LED 30, and microprocessor 31. Battery-charging connector 29 connects power from an outside source to charge battery 19. When power switch 26 is activated, battery 19 powers the plurality of UVC 280 nm LEDs 23 through the boost circuit 28 to boost the normal lithium-ion battery from 3.7 volts to 5 volts required to power the UVC LEDs. Microprocessor 31 detects power on, indicates green through the two-color status LED 30, and signals the audio transducer 27 for one beep. When the unit is off, microprocessor 31 signals the audio transducer 27 for two beeps. Microprocessor 31 also detects a low battery, turns off the green LED, turns on the Red LED and continuously beeps the audio transducer 27. During charging, the green LED flashes.

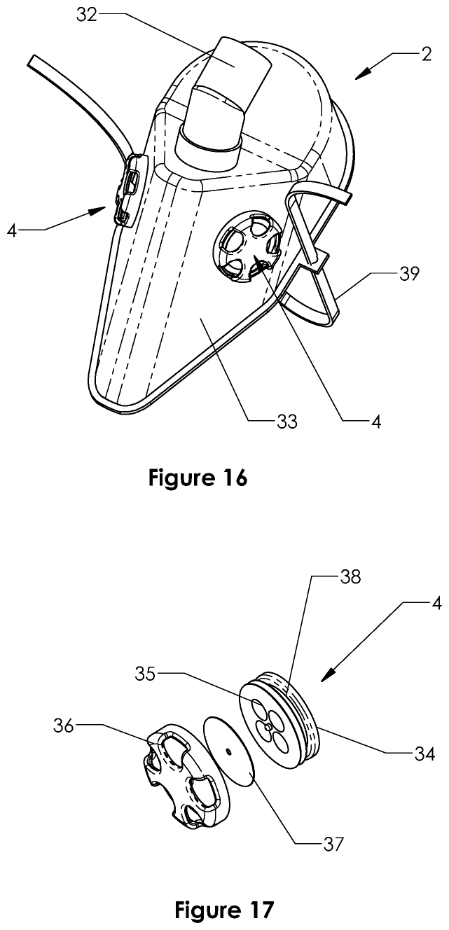

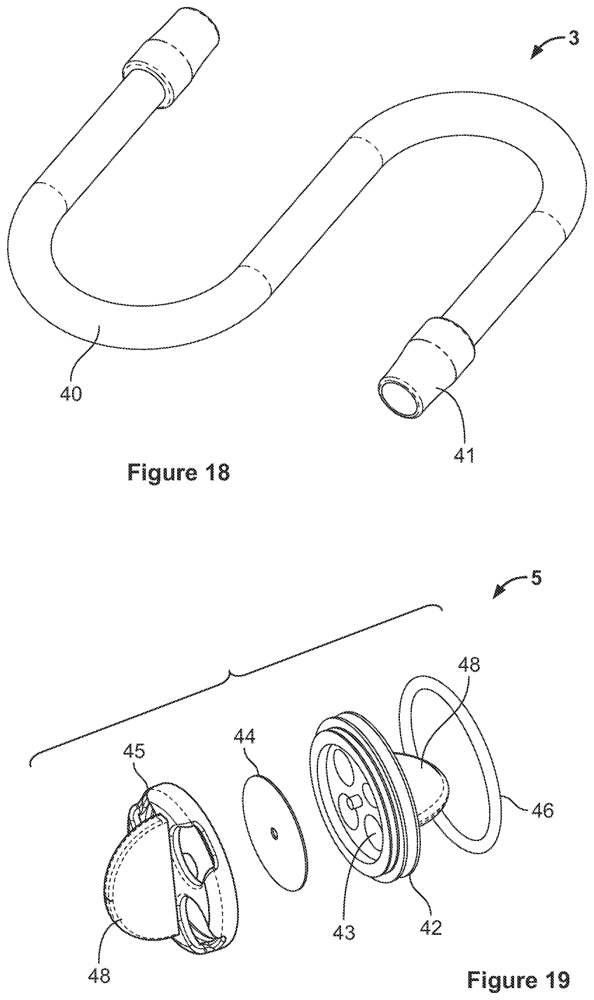

Referring to FIGS. 1, 16, 17, 18 and 20, hose assembly 3 consist of a breathing hose 40 and a urethane hose connector 41 on each end. One connector inserts into hose interface 17 on mainframe 8 and the other end slides onto the mask connector 32 on mask assembly 2. Mask assembly 2 consists of the mask 33, a soft PVC material that fits over the user's nose and mouth, an elastic strap 39 to secure mask to face, and two exhaust valve assemblies 4. The exhaust valve assemblies 4 consist of a mask valve body 34, mask valve cap 36 and mask valve disk 37. As pressure is decreased in the mask, during inhale, the mask valve disk 37 is drawn toward the low-pressure side through mask air aperture 35. The mask valve disk 37 is drawn tightly against valve body 34 and air flow is shut off. As pressure is increased in the mask, during exhale, the mask valve disk 37 is pushed away from valve body 34 and air flows freely. Valve mask groove 38 in mask valve body 34 allows exhaust valve assembly 4 to be installed in apertures in the mask 33.

Referring to FIGS. 3, 6, 7, 13 and 19, hose valve assembly 5 restricts or enables airflow from the filter to the mask, dependent on the installation direction of the valve by the user. Hose valve assembly 5 consist of a valve outlet cover 45, hose valve disk 44, valve inlet body 42 and O-ring 46. Tabs 48 on each side of the hose valve assembly 5 allow users to retrieve and reverse the hose valve assembly 5 in the hose interface 17 on mainframe 8. This changes air flow direction through the filter as desired by the user. O-ring 46 on valve inlet body 42 seals the hose valve assembly 5 in the hose interface 17 on mainframe 8. As pressure is increased on the valve inlet body 42 side of hose valve assembly 5, the hose valve disk 44 is pushed away from the valve inlet body 42 and air flows freely through the hose valve air apertures 43 in the valve inlet body 42. As pressure is increased on the valve outlet cover 45 side, the hose valve disk 44 is pushed toward the valve inlet body 42 and air flow is restricted through hose valve air apertures 43 in the valve inlet body 42.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

Parts List:
1 filter assembly
2 mask assembly
3 hose assembly
4 exhaust valve assembly
5 hose valve assembly
6 UV printed circuit board assembly
7 HEPA filter housing
8 mainframe
9 mainframe cover
10 apparel clip
11 filter housing clip
12 charge port cover
13 switch cover
14 seal boss
15 switch protection ring
16 screw
17 hose interface
18 HEPA filter
19 battery
20 printed circuit board
21 gasket
22 UV radiation
23 UVC 280 nm LED
24 UV apertures
25 clip notch
26 power switch
27 audio transducer
28 boost circuit
29 battery-charging connector
30 status LED
31 microprocessor
32 mask connector
33 mask
34 mask valve body
35 mask air aperture
36 mask valve cap
37 mask valve disk
38 mask valve groove
39 elastic strap
40 hose
41 hose connector
42 valve inlet body
43 hose air aperture
44 hose valve disk
45 valve outlet cover
46 O-ring
47 flow channel
48 tab

1. A reusable respirator comprising:
(a) a mask comprising an inlet and an exhaust valve, wherein the mask is configured to securely attach and seal to a user's face and to only emit from the exhaust valve carbon dioxide or other gases exhaled from the user during respiration and to receive oxygen through the inlet of the mask when the user inhales during respiration;
(b) a filter assembly positioned remotely from the mask, the filter assembly comprising:
(i) an enclosed housing comprising an inlet in contact with at least one of an external atmosphere or an external environment having oxygen therein and an outlet, the housing inlet and the housing outlet being in fluid connection with one another;
(ii) a replaceable filter comprising a vacuum high-efficiency particulate absorbing (HEPA) filter that is securely positioned completely over the inlet of the enclosed housing such that oxygen from at least one of the external environment or the external atmosphere passes through the replaceable vacuum HEPA filter internally into the enclosed housing towards the outlet of the enclosed housing during respiration of the user, the replaceable vacuum HEPA filter configured to at least one of filter or remove at least 99.95% of particles having greater than or equal to 0.3 μm diameter when passing oxygen internally into the enclosed housing from at least one of the external environment or the external atmosphere; and
(iii) an ultraviolet C (UVC) light source internally secured within the enclosed housing and positioned downstream of and directly above the replaceable vacuum HEPA filter such that the UVC light source, while in operation, sanitizes at least one of the replaceable vacuum HEPA filter or oxygen passing through the replaceable vacuum HEPA filter and flowing towards the outlet of the enclosed housing by emitting light within the UVC wavelength; and
(c) a flexible hose having two spaced apart ends with a first end of the flexible hose forming an airtight connection with the inlet of the mask and a second end of the flexible hose forming an airtight connection with the outlet of the enclosed housing of the filter assembly.

2. The reusable respirator of claim 1, wherein at least one of a connection or a contact interface between the replaceable vacuum HEPA filter and the inlet of the enclosed housing are sealed to prevent air leaks at least one of in or around the replaceable vacuum HEPA filter to prevent unfiltered oxygen from entering internally into the enclosed housing from at least one of the external environment or the external atmosphere.

3. The reusable respirator of claim 1, wherein the enclosed housing is openable and closable.

4. The reusable respirator of claim 1, wherein the enclosed housing further comprises a securing member that is configured to secure the enclosed housing to the user.

5. The reusable respirator of claim 1, wherein the UVC light source is operably connected to a power source.

6. The reusable respirator of claim 5, wherein the power source is rechargeable.

7. The reusable respirator of claim 5, wherein the power source is positioned internally within the enclosed housing.

8. (canceled)

9. A reusable respirator assembly comprising:
(a) a mask comprising an inlet and an exhaust valve;
(b) a filter assembly configured for remote positioning relative to the mask, the filter assembly comprising:
(i) an enclosed housing comprising an inlet configured for contact with at least one of an external atmosphere or an external environment having oxygen therein and an outlet, the housing inlet and housing outlet being in fluid connection with one another;
(ii) a replaceable filter comprising a vacuum high-efficiency particulate absorbing (HEPA) filter configured for at least one of secure positioning or attachment completely over the inlet of the enclosed housing such that, when assembled, oxygen from at least one of the external environment or the external atmosphere passes through the replaceable vacuum HEPA filter internally into the enclosed housing towards the outlet of the enclosed housing during respiration of a user, wherein the replaceable vacuum HEPA filter is configured to at least one of filter or remove at least 99.95% of particles having greater than or equal to 0.3 μm diameter when passing oxygen internally into the enclosed housing from at least one of the external environment or the external atmosphere; and
(iii) an ultraviolet C (UVC) light source internally secured within the enclosed housing and configured for positioning downstream of and directly above the replaceable vacuum HEPA filter, when fully assembled, such that the UVC light source is structured and arranged to sanitize at least one of the replaceable vacuum HEPA filter or oxygen passing through the replaceable vacuum HEPA filter and flowing towards the outlet of the enclosed housing by emitting light within the UVC wavelength; and
(c) a flexible hose having two spaced apart ends with the first end of the flexible hose configured for an airtight connection with the inlet of the mask and a second end of the flexible hose configured for an airtight connection with the outlet of the enclosed housing of the filter assembly.

10. The reusable respirator assembly of claim 9, wherein the enclosed housing is configured for at least one of a sealed connection or a sealed contact interface between the replaceable vacuum HEPA filter and the inlet of the enclosed housing, when assembled, to prevent air leaks at least one of in or around the replaceable vacuum HEPA filter to prevent unfiltered oxygen from entering internally into the enclosed housing from at least one of the external environment or the external atmosphere.

11. The reusable respirator assembly of claim 9, wherein the enclosed housing is openable and closable.

12. The reusable respirator assembly of claim 9, wherein the enclosed housing further comprises a securing member that is configured to secure the enclosed housing to the user.

13. The reusable respirator assembly of claim 9, wherein the UVC light source is operably connected to a power source.

14. The reusable respirator assembly of claim 13, wherein the power source is configured for recharging.

15. The reusable respirator assembly of claim 13, wherein the power source is positioned internally within the enclosed housing.

16. (canceled)
